(19) United States
(12) Patent Application Publication
(10) Pub. No.: US 2009/0263484 A1
(43) Pub. Date: Oct. 22, 2009

(54) Tissue Engineering Devices and Methods for Luminal Organs

(76) Inventors: Joseph J. Hammer, Hillsborough, NJ (US); Dhanuraj Shetty, Jersey City, NJ (US); Sridevi Dhanaraj, Raritan, NJ (US); Ziwei Wang, Monroe Twp., NJ (US); Jeffrey C. Geesin, Doylestown, PA (US); Daniel J. Keeley, Boston, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/107,408

(22) Filed: Apr. 22, 2008

(51) Int. Cl. A61K 9/00; A61K 47/30; A61K 38/46; A61K 38/55; A61K 35/38; A61K 31/00; A61P 43/00

(52) U.S. Cl. 424/486; 514/772.3; 424/94.62; 424/94.64; 424/94.67; 424/551; 424/93.7

(57) ABSTRACT

Tissue engineering devices and methods are provided for the reconstruction, repair, augmentation, or replacement of a luminal organ or tissue structure involving the use of a biodegradable polymer matrix conforming to a portion of a laminarly arranged luminal organ, the processing of autologous, allogeneic or xenogeneic tissue comprising multiple cell populations to obtain a minced tissue composition, the seeding of the matrix with the composition, and the implanting of the seeded polymer matrix into a patient.

### FIELD OF THE INVENTION

The present invention relates to methods and materials for tissue reconstruction, repair, augmentation, and replacement. More specifically, the present invention provides for the treatment of patients using an implantable device that is comprised of a biocompatible, biodegradable, synthetic or natural polymeric matrix shaped to conform to at least a part of a luminal organ or tissue structure and seeded with minced tissue.

### BACKGROUND

The human urinary bladder is a luminal organ constituting a musculomembranous sac situated in the anterior portion of the pelvic cavity. The bladder serves as a reservoir for urine, which this organ receives through the ureters and discharges through the urethra. In humans, the bladder is found in the pelvis behind the pelvic bone (pubis symphysis) and the urethra, which exits to the outside of the body. The bladder, ureters, and urethra are all similarly constituted in that they comprise muscular structures lined with a membrane comprising urothelial cells coated with mucus that is impermeable to the normal soluble substances of the urine. The trigone of the bladder (trigonum vesicae) is a smooth triangular portion of the mucous membrane at the base of the bladder. The bladder tissue is elastic and compliant, i.e., the bladder changes shape and size according to the amount of urine it contains. A bladder resembles a deflated balloon when empty, but becomes somewhat pear-shaped and rises into the abdominal cavity when the amount of urine increases.

The bladder wall has three main layers of tissues: the mucosa, submucosa, and detrusor. The mucosa, comprising urothelial cells, is the innermost layer and is composed of transitional cell epithelium. The submucosa lies immediately beneath the mucosa and its basement membrane. It is composed of blood vessels that supply the mucosa with nutrients and the lymph nodes, which aid in the removal of waste products. The detrusor is a layer of smooth muscle cells that expands to store urine and contracts to expel urine.

The bladder is subjected to numerous maladies and injuries that cause deterioration in patients. For example, bladder deterioration may result from infectious diseases, neoplasms, and developmental abnormalities. Bladder deterioration may also occur as a result of trauma from, for example, car accidents and sports injuries.

Although numerous biomaterials, including synthetic and naturally derived polymers, have been employed for tissue reconstruction or augmentation, no material has proven satisfactory for use in bladder reconstruction. Attempts have usually failed due to mechanical, structural, functional, or biocompatibility problems. Permanent synthetic materials have been associated with mechanical failure and calculus formation.

Naturally derived materials such as lyophilized dura, de-epithelialized bowel segments, and small intestinal submucosa have also been proposed for bladder replacement. However, it has been reported that bladder augmented with dura, peritoneum, and placenta and fascia contract over time. De-epithelialized bowel segments demonstrated an adequate urothelial covering for use in bladder reconstruction, but difficulties remain with mucosal regrowth, segment fibrosis, or both. It has been shown that de-epithelialization of the intestinal segments may lead to mucosal regrowth, whereas removal of the mucosa and submucosa may lead to retraction of the intestinal segment.

Other problems have been reported with the use of certain gastrointestinal segments for bladder surgery, including stone formation, increased mucus production, neoplasia, infection, metabolic disturbances, long-term contracture, and resorption. These attempts have demonstrated that it is not easy to replace the permeability functions of the urothelium.

Due to the multiple complications associated with the use of gastrointestinal segments for bladder reconstruction, alternate solutions have been sought. Recent surgical approaches have relied on native urological tissue for reconstruction, including auto-augmentation and ureterocystoplasty. However, auto-augmentation has been associated with disappointing long-term results and ureterocystoplasty is limited to cases in which a dilated ureter is already present. A system of progressive dilation for ureters and bladders has been proposed though not yet attempted clinically. Sero-muscular grafts and de-epithelialized bowel segments, either alone or over a native urothelium, have also been attempted. However, graft shrinkage and re-epithelialization of initially de-epithelialized bowel segments have been recurring problems.

One significant limitation besetting bladder reconstruction is directly related to the availability of donor tissue. The limited availability of bladder tissue prohibits the frequent routine reconstruction of bladder using normal bladder tissue. The bladder tissue that is available and considered usable may itself include inherent imperfections and disease. For example, in a patient suffering from bladder cancer, the remaining bladder tissue may be contaminated with metastasis. The patient is thus predestined to less than perfect bladder function.

Accordingly, a need exists in the art for improved methods and materials for the reconstruction, repair, augmentation, and replacement of luminal organs or tissue structures, such as the bladder. The deficiencies in the prior art are overcome by the present invention.

### SUMMARY OF THE INVENTION

An embodiment of the present invention relates to an organ reconstruction method comprising the steps of: providing a biodegradable polymer matrix conforming to a portion of a laminarly arranged luminal organ; obtaining autologous, allogeneic or xenogeneic tissue comprising multiple cell populations; processing the tissue to obtain a minced tissue composition; seeding the matrix with the composition; and implanting into a patient the seeded polymer matrix.

An embodiment of the present invention relates to an organ reconstruction method comprising the steps of: providing a biodegradable polymer matrix conforming to a portion of a laminarly arranged luminal organ; obtaining autologous, allogeneic or xenogeneic tissue comprising multiple cell populations; processing the tissue to obtain a first minced tissue composition and a second minced tissue composition; seeding a first area of the matrix with the first minced tissue composition, and seeding a second area of the matrix with the second minced tissue composition; and implanting into a patient the seeded polymer matrix.

Yet another embodiment of the present invention relates to an organ reconstruction device comprising an implantable, biodegradable polymer matrix conforming to a portion of a laminarly arranged luminal organ, wherein said matrix is capable of being seeded with a processed tissue composition, which comprises minced autologous, allogeneic or xenogeneic tissue comprising multiple cell populations.

### BRIEF DESCRIPTION OF THE DRAWINGS

Some features and advantages of the invention are described with reference to the drawings of certain preferred embodiments, which are intended to illustrate and not to limit the invention.

FIG. 1 depicts the anatomy of a normal human bladder.

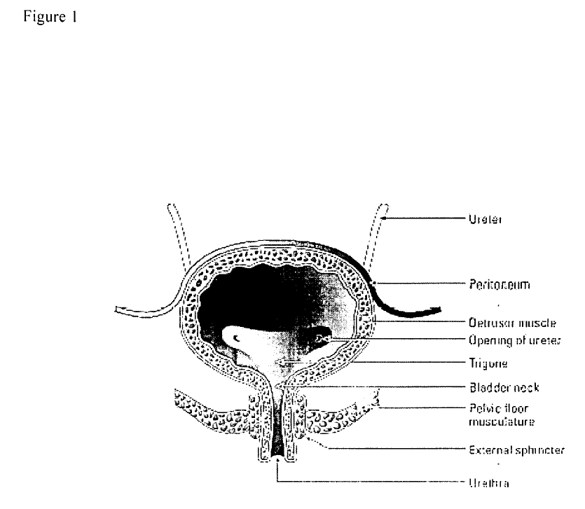

FIG. 2 depicts the tissue layers of various cell types that may be used in the minced tissue composition of the present invention.

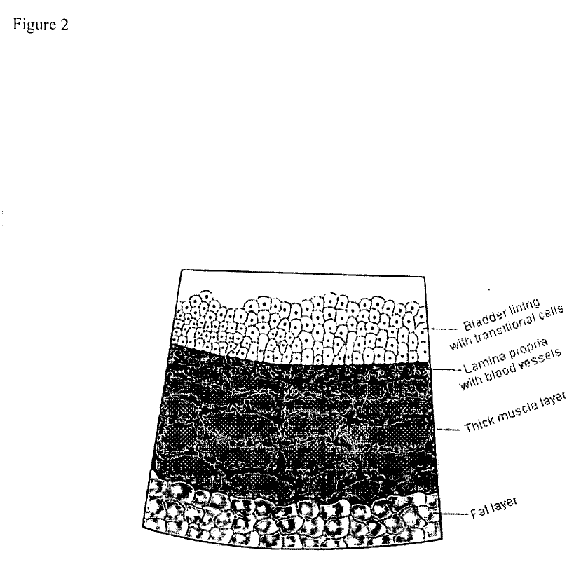

FIGS. 3A, 3B, and 3C depicts the cell migration, distribution and organization of urothelial and smooth muscle cells from bladder minced tissue into resorbable scaffolds. Arrows (1) point to urothelial cell clusters and layers; arrows (2) point to organization of smooth muscle like cells around the urothelial cells; and star denotes the cavity within the newly organized urothelium and smooth muscle structures.

### DETAILED DESCRIPTION OF THE INVENTION

It should be understood that this invention is not limited to the particular methodology, protocols, etc., described herein and, as such, may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Thus, for example, a reference to a cell may be a reference to one or more such cells, including equivalents thereof known to those skilled in the art unless the context of the reference clearly dictates otherwise. Unless defined otherwise, all technical terms used herein have the same meaning as those commonly understood to one of ordinary skill in the art to which this invention pertains. Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention provides for methods and materials for the reconstruction, repair, augmentation, or replacement of shaped hollow organs or tissue structures that exhibit a laminar segregation of different cell types and that have a need to retain a general luminal shape. Luminal organs or tissue structures containing a smooth muscle cell layer to impart compliant or contractible properties to the organ or structure are particularly well-suited to the methods and devices of the present invention.

One example of a luminal organ suitable for application of the present invention is a bladder, which has an inner layer of a first cell type that comprises urothelial-tissue, a middle layer of submucosa, and an outer layer of a second cell type that comprises smooth muscle tissue. This organization is also present in other genitourinary organs and tissue structures such as the renal pelvis ureters and urethra. Laminarily organized organs or tissues refer to any organ or tissue made up of, or arranged in laminae, including ductal tissue. Other suitable laminarily organized luminal organs, tissue structure, or ductal tissues to which the present invention is directed include vas deferens, fallopian tubes, lacrimal ducts, trachea, stomach, intestines, vasculature, biliary duct, ductus ejaclatoruis, ductus epididymidis, ductus parotideus, ureters, urethras, and surgically created shunts.

The present invention may be suitable for the treatment of such conditions as bladder extrophy, bladder volume insufficiency, reconstruction of bladder following partial or total cystectomy, repair of bladders damaged by trauma, and the like.

While reference is made herein to the reconstruction, repair, augmentation, and replacement of the bladder, it will be understood that the methods and devices of the invention are useful for the reconstruction, repair, augmentation, and replacement of a variety of tissues and organs in a patient. Thus, for example, organs or tissues such as bladder, ureter, urethra, renal pelvis, and the like, can be reconstructed, repaired, augmented, or replaced with polymeric matrixes seeded with the appropriate minced tissue. The devices and methods of the invention can be further applied to the reconstruction, repair, augmentation, and replacement of vascular tissue (see, e.g., Zdrahala, R. J., J. Biomater. Appl. 10(4): 309-29 (1996)), intestinal tissues, stomach (see, e.g., Laurencin, C. T. et al., J. Biomed. Mater. Res. 30(2): 133-38 (1996)), and the like. The patient to be treated may be of any species of mammals, such as a dog, cat, pig, horse, cow, or human, in need of reconstruction, repair, augmentation, or replacement of an organ or tissue structure.

The source of the minced tissue of the present invention may be of the same or different tissue origin than that intended to be reconstructed, repaired, augmented, and replaced. For example, the minced tissue may derive from urethral tissue to facilitate the reconstruction, repair, augmentation, and replacement of bladder tissue. The morphologic similarity of luminal organs, such as bladder and urethral tissue, for example, is known in the art, see Dass et al., 165 J. Urol. 1294-1299 (2001), and the use of bladder tissue in urethra reconstruction has been reported, A. Atala, 4 (Suppl. 6) Am. J. of Transplantation 5873 (2004).

As stated earlier, one significant limitation besetting bladder reconstruction is directly related to the availability of donor tissue. The limited availability of bladder tissue prohibits the frequent routine reconstruction of bladder using normal bladder tissue. The bladder tissue that is available and considered usable may itself include inherent imperfections and disease. For example, in a patient suffering from bladder cancer, the remaining bladder tissue may be contaminated with metastasis. The patient is thus predestined to less than perfect bladder function.

As a result, others have tried a cell culturing approach (Atala et al.) where the smooth muscle cells and the urothelium cells are isolated from a biopsy, cultured separately in vitro, and then added onto a bladder substrate. However, this process is long and time consuming where a patient has to wait for at least eight weeks before the next implantation of a tissue engineered scaffold. Other tissues have also been evaluated as a source of cells for bladder augmentation for buccal tissue, for example. See El-Sherbiny et al., “Treatment of Urethral Defects Skin, Buccal or Bladder Mucosa, Tube or Patch? An Experimental Study in Dogs,” 167 J. Urol. 2225-2228 (2002).

The methods of the present invention provide a biocompatible synthetic or natural polymeric matrix that is shaped to conform to its use as a part or all of the bladder structure to be repaired, reconstructed, augmented or replaced. A biocompatible material is any substance not having toxic or injurious effects on biological function. As used herein the term “synthetic polymer” refers to polymers that are not found in nature, even if the polymers are made from naturally occurring biomaterials. The term “natural polymer” refers to polymers that are naturally occurring. The shaped, synthetic or natural polymeric matrix is preferably porous to allow for cell deposition and migration both on and in the pores of the matrix. It can be made from various scaffolding materials such as lyophilized foams, nonwoven scaffolds, or melt-blown scaffolds.

Lyophilization, or freeze-drying, removes a solvent from a polymer-solvent solution through sublimation, leaving behind a porous solid. More specifically, the process separates a solvent from a frozen solution through a solid to gas phase transition. This transition, called sublimation, removes the solvent without it ever entering a liquid state. The final construct is a porous solid structure made out of the remaining solute often described as a foam.

Liquid solution comprising any natural or synthetic biocompatible, biodegradable polymer, or any blend of such polymers, dissolved in a solvent that can be removed through sublimation, is poured into an open-ended, hinged mold and mechanically rotated during freezing. In the first step, the mold is hinged shut and partially filled with solution. During filling, some of the mold's volume remains empty. After lyophilization, the volume of solution poured into the mold will make up the scaffold volume whereas the empty volume will make up the hollow void. After filling, the mold may be rotated in a number of ways. When the mold is held vertically and spun quickly, a centrifugal force acts on the liquid solution, pushing it away from the mold's center and up upon its sides. The spinning mold may then be cooled slowly or flash frozen by submersion in liquid nitrogen. The mold may also be held horizontally and rotated slowly whereby gravity allows the polymer to settle upon one side of the mold. Assuming that the temperature of the mold is lower than the temperature of the ambient air, a layer of frozen liquid will gradually build up on the mold's interior, resulting in an internal frozen skin. Both methods will produce a frozen construct that has a shape and texture consistent with the mold's internal geometry. Once fully frozen, the construct is placed in a vacuum for sublimation.

A variety of absorbable polymers can be used to make foams. Examples of suitable biocompatible, bioabsorbable polymers that could be used include polymers selected from the group consisting of aliphatic polyesters, poly(amino acids), copoly(ether-esters), polyalkylene oxalates, polyamides, poly(iminocarbonates), polyorthoesters, polyoxaesters, polyamindoesters, polyoxaesters containing amine groups, poly(anhydrides), polyphosphzenes, biomolecules (i.e., biopolymers such as collagen, elastin, bioabsorbable starches, etc.), and blends thereof.

Suitable solvents include but are not limited to solvents selected from a group consisting of formic acid, ethyl formate, acetic acid, hexafluoroisopropanol (HFIP), cyclic ethers (i.e., THF, DMF, and PDO), acetone, acetates of C₂ to C₅ alcohol (such as ethyl acetate and t-butylacetate), glyme (i.e., monoglyme, ethyl glyme, diglyme, ethyl diglyme, triglyme, butyl diglyme, and tetraglyme), methylethyl ketone, dipropyleneglycol methyl ether, lactones (such as γ-valerolactone, δ-valerolactone, β-butyrolactone, γ-butyrolactone), 1,4-dioxane, 1,3-dioxolane, 1,3-dioxolane-2-one (ethylene carbonate), dimethylcarbonate, benzene, toluene, benzyl alcohol, p-xylene, naphthalene, tetrahydrofuran, N-methylpyrrolidone, dimethylformamide, chloroform, 1,2-duchloromethane, morpholine, dimethylsulfoxide, hexafluoroacetone sesquihydrate (HFAS), anisole and mixtures thereof. A homogenous solution of the polymer in the solvent is prepared using standard techniques.

As will be appreciated by those skilled in the art, the applicable polymer concentration or amount of solvent which may be utilized will vary with each system. Suitable phase diagram curves for several systems have already been developed. However, if an appropriate curve is not available, this can be readily developed by known techniques. The amount of polymer will depend to a large extent on the solubility of the polymer in a given solvent and the final properties of the foam desired.

A parameter that may be used to control foam structure is the rate of freezing of the polymer-solvent solution. The type of pore morphology that gets locked in during the freezing step is a function of the solution thermodynamics, freezing rate, temperature to which it is cooled, concentration of the solution, homogeneous or heterogeneous nucleation, etc. Detailed description of such phase separation phenomenon can be found in the references provided herein. See A. T. Young, “Microcellular foams via phase separation,” J. Vac. Sci. Technol. A 4(3), May/June 1986; S. Matsuda, “Thermodynamics of Formation of Porous Polymeric Membrane from Solutions,” Polymer J. Vol. 23, No. 5, pp 435-444, 1991).

A foam scaffold may also be constructed by a two-step mold where one part of the mold consists of a hollow section and another part consists of a core. This design is similar to that used in a typical injection molding process. The solution can be filled via the space between the cavity and the core. The space can be determined by the thickness of the final construct. Once the filling is complete, the solution can be frozen by the steps above.

Another embodiment of the present invention may include nonwoven scaffolds. Preferred nonwoven materials include flexible, porous structures produced by interlocking layers or networks of fibers, filaments, or film-like filamentary structures. Such nonwoven materials can be formed from webs of previously prepared/formed fibers, filaments, or films processed into arranged networks of a desired structure.

Generally, nonwoven materials are formed by depositing the constituent components (usually fibers) on a forming or conveying surface. These constituents may be in a dry wet, quenched, or molten state. Thus, the nonwoven can be in the form of a dry laid, wet laid, or extrusion-based material, or hybrids of these types of nonwovens can be formed. The fibers or other materials from which the nonwovens can be made are typically polymers, either synthetic or naturally occurring.

Dry laid scaffolds may include those nonwovens formed by garneting, carding, and/or aerodynamically manipulating dry fibers in the dry state. In addition, wet laid nonwovens may be formed from a fiber-containing slurry that is deposited on a surface, such as moving conveyor. The nonwoven web can be formed after removing the aqueous component and drying the fibers. Extrusion-based nonwovens may include those formed from spunbond fibers, melt blown fibers, and porous film systems. Hybrids of these nonwovens can be formed by combining one or more layers of different types of nonwovens by a variety of lamination techniques. The nonwoven may also be reinforced with a woven, knit or mesh fabric.

The nonwovens of the present invention preferably have a density designed to obtain mechanical characteristics ideal for augmenting bladder repair. The density may be measured by determining the felt dimensions (length and width), for example, obtaining two measurements in each direction to calculate the average length and width for each nonwoven felt. The trimmed felt may be weighed, and the weight recorded. The average thickness of each nonwoven felt may be obtained using a Shirley gauge. The density may be calculated by the following formula:

Density=(weight of felt(W)(grams))/(length×width (cm²))=((W×1000 (mg/cm²))/((thickness (mm))/10 (mm/cm))

Additionally, scaffolds may be manufactured by use of melt-blowing technology whereby fibrous webs from molten polymer resin are extruded from spinnarettes onto a rotating collapsible object in the presence of a porogen. The collapsible object can be made to rotate or otherwise move therefore allowing a coating of extruded polymer to layer itself substantially evenly on the collapsible object. Continuous rotation of the surface will produce an increasingly thick or dense layer due to more polymer being deposited. The use of a collapsible object creates seamless, three-dimensional shapes of polymer web. Specifically, the final product may be a hollow shape with a single outlet from which the collapsed shape has been removed. More complex geometries may be achieved by using suitably shaped tooling such as a mold or mandrel to guide the formation of the melt-blown filaments into a specific shape. This method is described in detail by Keeley et al. in U.S. patent application Ser. No. 11/856,743.

Melt-blown technology is able to incorporate synthetic biopolymers, such as PGA, PLA or their respective copolymers, and natural polymers. A scaffold constructed of either material is both biocompatible and resorbable but may not be sufficiently porous to facilitate optimal proliferation of cells or advanced tissue ingrowth. To overcome this obstacle, a porogen may be added during the fabrication of the non-woven web. Porogens such as salt or glucose spheres can be dusted or blown onto the molten fibers during their extrusion. Gelatin microspheres can also be used. The resulting scaffold's porosity can be controlled by the amount of porogen added, while the pore size is dependent on the size of the spheres. As these particles enter the turbulent air, they are randomly incorporated into the web. Because the filaments in the melt-blown structure will typically shrink due to crystallization as they age, the porous structure may undergo an annealing process with the porogen material in place. Once the porogen-fiber composite is annealed, the entire construct may then be submerged in water so that the porogens dissolve or leach out of the web. The resulting matrix contains polymer fibers but with increased distance between them to effect porosities. In one embodiment, the matrix has more porogen and hence, more porosity, the porosity in excess of 90%.

The polymers or polymer blends that are used to form the biocompatible, biodegradable scaffold may also contain pharmaceutical compositions. The previously described polymer may be mixed with one or more pharmaceutical prior to forming the scaffold. Alternatively, such pharmaceutical compositions may coat the scaffold after it is formed. The variety of pharmaceuticals that can be used in conjunction with the scaffolds of the present invention includes any known in the art. In general, pharmaceuticals and/or biologics that may be administered via the compositions of the invention include, without limitation: anti-infectives such as antibiotics and antiviral agents; chemotherapeutic agents; anti-rejection agents; analgesics and analgesic combinations; anti-inflammatory agents; hormones such as steroids; growth factors; and other naturally derived or genetically engineered (recombinant) proteins, polysaccharides, glycoproteins, or lipoproteins.

Scaffolds containing these materials may be formulated by mixing one or more agents with the polymer used to make the scaffold or with the solvent or with the polymer-solvent mixture. Alternatively, an agent could be coated onto the scaffold, preferably with a pharmaceutically acceptable carrier. Any pharmaceutical carrier may be used that does not substantially degrade the scaffold. The pharmaceutical agents may be present as a liquid, a finely divided solid, or any other appropriate physical form. Typically, but optionally, they will include one or more additives, such as diluents, carriers, excipients, stabilizers or the like. In addition, various biologic compounds such as antibodies, cellular adhesion factors, growth factors, and the like, may be used to contact and/or bind delivery agents of choice (e.g., pharmaceuticals or other biological factors) to the scaffold of the present invention.

Synthetic polymers can also be modified in vitro before use, and can carry growth factors and other physiologic agents such as peptide and steroid hormones, which promote proliferation and differentiation. The polyglycolic acid polymer undergoes biodegradation over a four month period; therefore as a cell delivery vehicle it permits the gross form of the tissue structure to be reconstituted in vitro before implantation with subsequent replacement of the polymer by an expanding population of engrafted cells.

The polymeric matrix may be shaped into any number of desirable configurations to satisfy any number of overall systems, geometries, or space restrictions. For example, in the use of the polymeric matrix for bladder reconstruction, the matrix may be shaped to conform to the dimensions and shapes of the whole or a part of a bladder. Furthermore, the polymeric matrix may be shaped in different sizes and shapes to conform to the bladders of differently sized patients. Optionally, the polymeric matrix should be shaped such that after its biodegradation, the resulting reconstructed bladder may be collapsible when empty in a fashion similar to a natural bladder. The polymeric matrix may also be shaped in other fashions to accommodate the special needs of the patient. For example, a previously injured or disabled patient may have a different abdominal cavity and may require a bladder reconstructed to adapt to fit it. Furthermore, the portion of a laminarly arranged luminal organ to which the polymeric matrix can be conformed may be relatively minor. For example, 70% to 80%, or more, of the luminal organ could be replaced using the methods and materials of the present invention.

Recent publications have discussed seeding a supporting matrix with cells for purposes of tissue regeneration in such organs as the bladder. A. Atala, in “Tissue Engineering for Bladder Substitution,” World J. Urol. 18: 364-70, 365 (2000), refers to techniques all involving the use of “cells that are dissociated and expanded in vitro, reattached to a matrix, and implanted.” Specifically, the article describes a “system . . . which does not use any enzymes or serum and has a large expansion potential.” J. Yoo et. al., in “Bladder Augmentation Using Allogeneic Bladder Submucosa Seeded with Cells,” Urology 51:221-225 (1998), used urothelial and smooth muscle cells that were harvested and expanded from dog to seed allogeneic bladder submucosa. U.S. Pat. No. 6,576,019 discloses methods and devices involving “cell populations” that have been isolated and cultured in vitro to increase the number of cells available for seeding. These approaches are not based on directly seeding a polymeric matrix with use minced tissue that has not been cultured in vitro. Patent No. EP1410811 discusses the use of minced tissue to seed a biocompatible scaffold for purposes of repairing and or regenerating diseased or damaged tissue. Nowhere in the patent, however, is the invention applied to the regeneration of full organs.

The polymeric matrix of the present invention includes a biocompatible scaffold having at least a portion in contact with a minced tissue suspension. The minced tissue suspension can be disposed on the outer surface of the scaffold, on an inner region of the scaffold, and any combination thereof, or alternatively, the entire scaffold can be in contact with the minced tissue suspension.

The tissue can be obtained using any of a variety of conventional techniques, such as for example, by biopsy or surgical removal. Preferably, the tissue sample is obtained under aseptic conditions. Once a sample of living tissue has been obtained, the sample can then be processed under sterile conditions to create a suspension having at least one minced, or finely divided, tissue particle. The particle size and shape of each tissue fragment can vary, for example, the tissue size can be in the range of about 0.1 and 3 mm³, in the range of about 0.5 and 1 mm³, in the range of about 1 to 2 mm³, or in the range of about 2 to 3 mm³, but preferably the tissue particle is less than 1 mm³. The shape of the tissue fragments can include slivers, strips, flakes or cubes as examples. Some methods include mechanical fragmentation or optical/laser dissections.

The tissue samples used in the present invention are obtained from a donor (autogeneic, allogeneic, or xenogeneic) using appropriate harvesting tools. The tissue samples can be finely minced and divided into small particles either as the tissue is collected, or alternatively, after it is harvested and collected outside the body. Mincing the tissue can be accomplished by a variety of methods. In one embodiment, the mincing is accomplished with two sterile scalpels using a parallel direction, and in another embodiment, the tissue can be minced by a processing tool that automatically divides the tissue into particles of a desired size. In one embodiment, the minced tissue can be separated from the physiological fluid and concentrated using any of a variety of methods known to those having ordinary skill in the art, such as for example, sieving, sedimenting or centrifuging. In embodiments where the minced tissue is filtered and concentrated, the suspension of minced tissue preferably retains a small quantity of fluid in the suspension to prevent the tissue from drying out. In another embodiment, the suspension of minced tissue is not concentrated, and the minced tissue can be directly delivered to the site of tissue repair via a high concentration tissue suspension or other carrier such as for example, a hydrogel, fibrin glue, or collagen. In this embodiment, the minced tissue suspension can be covered by any of the biocompatible scaffolds described above to retain the tissue fragments in place.

The minced tissue can then be distributed onto a scaffold using a cell spreader or other tools known in the art. The minced tissue can be dispersed onto a scaffold in one of several ways. In one example, a biopsy of tissue sample comprising of full thickness of the bladder can be obtained. Tissue can be minced as a whole and distributed on the scaffold. In a second example, a partial thickness biopsy of tissue sample can be obtained and minced as a whole and distributed on the scaffold. The difference in these two methods is the proportion of the urothelial cells to other cells, for example, smooth muscle cells. A third example includes separating the urothelial layer and seromuscular layer and subsequently mincing the layers separately before distributing each onto to surfaces of the scaffold. In a fourth example, the urothelial minced tissue can be distributed on a scaffold seeded with isolated smooth muscle cells. In a fifth example, the minced smooth muscle tissue can be combined with a scaffold seeded with isolated urothelial cells. In a sixth example, the urothelial and or smooth muscle minced tissue can be combined with stem cells seeded on the scaffold.

The minced tissue has at least one viable cell that can migrate from the tissue fragment onto the scaffold. The tissue contains an effective amount of cells that can migrate from the tissue fragment and begin populating the scaffold. In one embodiment, the minced tissue particles can be formed as a suspension in which the tissue particles are associated with a physiological buffering solution. Suitable physiological buffering solutions include, but are not limited to, saline, phosphate buffer solution, Hank's balanced salts, Tris buffered saline, Hepes buffered saline and combinations thereof. In addition the tissue can be minced in any standard cell culture medium known to those having ordinary skill in the art, either in the presence or absence of serum. Prior to depositing the suspension of minced tissue on the scaffold or at the site of tissue/organ injury, the minced tissue suspension can be filtered and concentrated, such that only a small quantity of physiological buffering solution remains in the suspension.

The minced tissue fragments may be contacted with a matrix-digesting enzyme to facilitate cell migration out of the extracellular matrix and into the scaffold material. Suitable matrix-digesting enzymes that can be used in the present invention include, but are not limited to, collagenase, chondroitinase, trypsin, elastase, hyaluronidase, peptidase, thermolysin, and protease.

### EXAMPLE

### Example 1

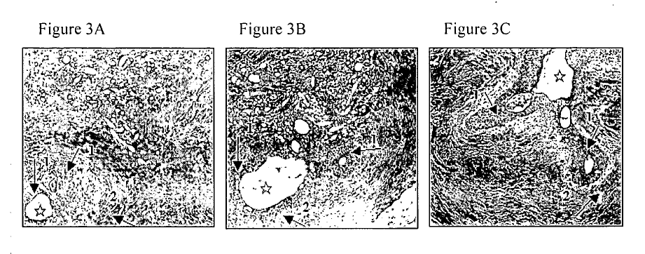

Healthy intact bladder tissue was be obtained from a porcine source. The bladder tissue was dissected open, and intravesicular fluid within the bladder was aspirated out. The bladder tissue was then rinsed three times with phosphate buffered saline (PBS), and partial thickness biopsies were obtained from the bladder consisting of the urothelium layer, submucosa and a portion of the smooth muscle layer. The biopsied tissue was minced to a fine paste. This tissue paste was then distributed evenly on a 5 mm punch of bioresorbable scaffold such that the minced tissue paste completely covered the scaffold. The scaffold loaded with minced tissue was implanted subcutaneously into severe combined immunodeficiency (SCID) mice for 4 weeks. Hematoxylin and Eosin (H/E) stained histological sections were analyzed for cell migration, distribution and organization within and around the scaffolds, and for the nature of matrix formed. FIG. 3 shows the extent of cell migration into the polymer scaffolds from the minced bladder tissue fragments. Clusters of urothelial cells are observed surrounded by smooth muscle cells. The size of the organized clusters range from small ones with central urothelial clusters (FIG. 3A), to larger ones with a central cavity (FIG. 3B). As these clusters grew they also began to coalesce to form a larger structure (FIG. 3C) with well organized urothelial cell layers surrounded by smooth muscle like cell layer with a central cavity. These structures resemble the organization seen in typical normal bladder. These figures demonstrate that the cells are able to migrate from the minced tissue into the scaffolds and are able to segregate and reorganize themselves into bladder like structures.

We claim:

1. An organ reconstruction method comprising the steps of:
providing a biodegradable polymer matrix conforming to a portion of a laminarly arranged luminal organ;
obtaining autologous, allogeneic or xenogeneic tissue comprising multiple cell populations;
processing the tissue to obtain a minced tissue composition;
seeding the matrix with the composition; and
implanting into a patient the seeded polymer matrix.

2. The method of claim 1 further comprising the step of adding one or more matrix-digesting enzymes to said minced tissue composition.

3. The method of claim 2 wherein said matrix-digesting enzyme is selected from the group consisting of collagenase, chondroitinase, trypsin, elastase, hyaluronidase, peptidase, thermolysin, protease, and combinations thereof.

4. The method of claim 1 further comprising the step of adding one or more pharmaceuticals to said minced tissue composition.

5. The method of claim 4 wherein said pharmaceutical is selected from the group consisting of antibiotics, antiviral agents, chemotherapeutic agents, anti-rejection agents, analgesics, anti-inflammatory agents, hormones, steroids, growth factors, naturally derived proteins, genetically engineered proteins, polysaccharides, glycoproteins, lipoproteins, and combinations thereof.

6. The method of claim 1 further comprising the step of adding one or more pharmaceuticals to said polymer matrix.

7. The method of claim 6 wherein said pharmaceutical is selected from the group consisting of antibiotics, antiviral agents, chemotherapeutic agents, anti-rejection agents, analgesics, anti-inflammatory agents, hormones, steroids, growth factors, naturally derived proteins, genetically engineered proteins, polysaccharides, glycoproteins, lipoproteins, and combinations thereof.

8. The method of claim 1 further comprising the step of adding one or more stem cells to said minced tissue composition.

9. An organ reconstruction method comprising the steps of:
providing a biodegradable polymer matrix conforming to a portion of a laminarly arranged luminal organ;
obtaining autologous, allogeneic or xenogeneic tissue comprising multiple cell populations;
processing the tissue to obtain a first minced tissue composition and a second minced tissue composition;
seeding a first area of the matrix with the first minced tissue composition, and seeding a second area of the matrix with the second minced tissue composition; and
implanting into a patient the seeded polymer matrix.

10. The method of claim 9 wherein said first minced tissue composition is comprised of a smooth muscle tissue and said second minced tissue composition is comprised of an endothelial tissue.

11. The method of claim 9 further comprising the step of adding one or more matrix-digesting enzymes to one or more minced tissue compositions.

12. The method of claim 11 wherein said matrix-digesting enzyme is selected from the group consisting of collagenase, chondroitinase, trypsin, elastase, hyaluronidase, peptidase, thermolysin, protease, and combinations thereof.

13. The method of claim 9 further comprising the step of adding one or more stem cells to one or more minced tissue compositions.

14. An organ reconstruction device comprising an implantable, biodegradable polymer matrix conforming to a portion of a laminarly arranged luminal organ, wherein said matrix is capable of being seeded with a processed tissue composition, which comprises minced autologous, allogeneic or xenogeneic tissue comprising multiple cell populations.

15. The device of claim 14 further comprising a polymer mesh fabric.

16. The device of claim 14 further comprising a one or more pharmaceuticals.

17. The device of claim 16 wherein said pharmaceutical is selected from the group consisting of antibiotics, antiviral agents, chemotherapeutic agents, anti-rejection agents, analgesics, anti-inflammatory agents, hormones, steroids, growth factors, naturally derived proteins, genetically engineered proteins, polysaccharides, glycoproteins, lipoproteins, and combinations thereof.

18. A reinforced organ reconstruction device comprising an implantable, biocompatible polymer mesh having a first surface and a second surface, further having a first biodegradable polymer matrix in contact with said first polymer mesh surface, and further having a second biodegradable polymer matrix in contact with said second polymer mesh surface, wherein said polymer matrix-mesh-matrix construct is conforming to a portion of a laminarly arranged luminal organ, and further wherein said first and second polymer matrices are capable of being seeded with a processed tissue composition, which comprises minced autologous, allogeneic or xenogeneic tissue comprising multiple cell populations.

19. The device of claim 18 further comprising a pharmaceutical.

20. The device of claim 19 wherein said pharmaceutical is selected from the group consisting of antibiotics, antiviral agents, chemotherapeutic agents, anti-rejection agents, analgesics, anti-inflammatory agents, hormones, steroids, growth factors, naturally derived proteins, genetically engineered proteins, polysaccharides, glycoproteins, lipoproteins, and combinations thereof.
